(19) United States
(12) Patent Application Publication
(10) Pub. No.: US 2005/0203174 A1
(43) Pub. Date: Sep. 15, 2005

(54) Combination therapies using leptomycin B

(75) Inventors: Daniel V. Santi, San Francisco, CA (US); Yiqing Zhou, Lafayette, CA (US)

Correspondence Address: KOSAN BIOSCIENCES, INC, 3832 BAY CENTER PLACE, HAYWARD, CA, 94588, US

(73) Assignee: Kosan Biosciences, Inc., Hayward, CA (US)

(21) Appl. No.: 11/058,447

(22) Filed: Feb. 14, 2005

Related U.S. Application Data
(60) Provisional application No. 60/551,970, filed on Mar. 9, 2004.

(51) Int. Cl. A61K 31/366; A61K 31/7072; A61K 31/704; A61K 31/4745; A61K 31/337

(52) U.S. Cl. 514/460; 514/575; 514/492; 514/410; 514/365; 514/34; 514/283; 514/406; 514/269; 514/49

(57) ABSTRACT

Diseases of cellular proliferation can be treated with a combination of leptomycin B and a chemotherapeutic co-agent, for instance an anti-mitotic agent, a DNA cleaver, an alkylating agent, a DNA crosslinking agent, a DNA intercalator, an HSP90 inhibitor, a topoisomerase I inhibitor, a topoisomerase II inhibitor, an immunosuppressant, an anti-metabolite, a COX-2 inhibitor, a nucleoside (purine or pyrimidine) analog, a Ras inhibitor, a farnesyl transferase inhibitor, or a histone deacetylase inhibitor.

### CROSS-REFERENCE TO RELATED APPLICATIONS

This application claims the benefit of U.S. Provisional Application No. 60/551,970, filed Mar. 9, 2004; the disclosure of which is incorporated herein by reference.

### BACKGROUND OF THE INVENTION

1. Field of the Invention

This invention relates to anti-tumor treatments using a combination of leptomycin B and a co-agent.

2. Description of Related Art

Leptomycin B (also referred to as LMB) is an anti-tumor, anti-microbial substance originally isolated from various Steptomyces strains. Hokanson et al., U.S. Pat. No. 4,771,070 (1988); Nettleton et al., U.S. Pat. No. 4,792,522 (1988).

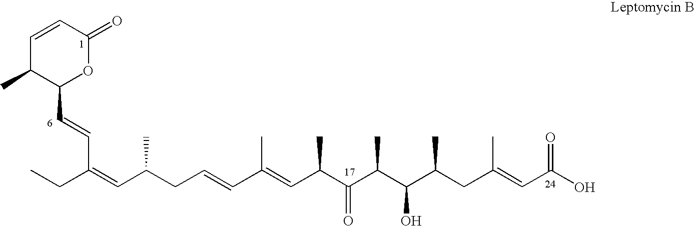

At the cellular level, leptomycin B has been shown to act by arresting cells at the end of the G1 and G2 phases of the cell cycle. At the molecular level, leptomycin B acts as an inhibitor of the nuclear export receptor CRM1, which binds to and effects the nuclear translocation of “cargo proteins” such as P53, STAT1, (i)ADAR1, Rev, actin, and Bcr-abl. Nishi et al., J. Biol. Chem., 269 (9), 6320-6324 (1994); Fukuda et al., Nature 390, 308-311 (1997). These observations have lead to interest in leptomycin B as an anti-cancer agent. See, e.g., Wang et al., U.S. 2003/0162740 A1 (2003).

### BRIEF SUMMARY OF THE INVENTION

In a first aspect of the invention, there is provided a method of treating a disease of cellular proliferation, comprising administering to a subject in need of such treatment a therapeutically effective amount of a combination of leptomycin B and a chemotherapeutic co-agent.

In a preferred embodiment, the chemotherapeutic co-agent is selected from the group consisting of an anti-mitotic agent, a DNA cleaver, an alkylating agent, a DNA crosslinking agent, a DNA intercalator, an HSP90 inhibitor, a topoisomerase I inhibitor, a topoisomerase II inhibitor, an immunosuppressant, an anti-metabolite, a COX-2 inhibitor, a nucleoside (purine or pyrimidine) analog, a Ras inhibitor, a farnesyl transferase inhibitor, and a histone deacetylase inhibitor.

In another preferred embodiment, the chemotherapeutic co-agent is selected from the group consisting of altretamine, busulfan, oxaliplatin, thiotepa, irinotecan, bleomycin, doxorubicin, mitomycin, fludarabine, fluorouracil, gemcitabine, aminoglutethimide, bicalutamide, celecoxib, L-744832, SAHA, docetaxel, epothilone D, vinblastine, gefitinib, trastuzumab, 17-AAG, paclitaxel, imatinib, methotrexate, capecitabine, vincristine, hydroxyurea, vindesine, FK-506, rapamycin, trichostatin A, callystatin A, cisplatin, and discodermolide.

In particular embodiments, the disease of cellular proliferation is cancer, especially colon cancer.

In one embodiment, the leptomycin B and the chemotherapeutic co-agent are administered simultaneously. In another embodiment, the leptomycin B is administered before the chemotherapeutic co-agent. In yet another embodiment, the chemotherapeutic co-agent is administered before the leptomycin B.

### DETAILED DESCRIPTION OF THE INVENTION

The present invention relates to combination therapies involving a combination of leptomycin B and a chemotherapeutic co-agent. In a first embodiment, the leptomycin B and chemotherapeutic co-agent are administered simultaneously to a patient suffering from a disease of cellular proliferation, in particular cancer. In a second embodiment, the chemotherapeutic co-agent is administered first to such patient, followed by leptomycin B. In a third embodiment, the leptomycin B is administered first to such patient, followed by the chemotherapeutic co-agent. Depending on the administration regimen and the chemotherapeutic co-agent, different levels of efficacy of the combination treatment were attained.

In one embodiment, the chemotherapeutic co-agent is a cytotoxic drug. In another embodiment, the chemotherapeutic co-agent is selected from the group consisting of an anti-mitotic agent, a DNA cleaver, an alkylating agent, a DNA crosslinking agent, a DNA intercalator, an HSP90 inhibitor, a topoisomerase I inhibitor, a topoisomerase II inhibitor, an immunosuppressant, an anti-metabolite, a COX-2 inhibitor, a nucleoside (purine or pyrimidine) analog, a Ras inhibitor, a farnesyl transferase inhibitor, a histone deacetylase inhibitor, and the like.

Specific suitable chemotherapeutic co-agents include altretamine (hexamethylmelamine, Hexalen™), busulfan (Busulfex™, Myleran™), oxaliplatin (Eloxatin™), thiotepa (triethylenethiophosphoramide, Tespamin™, Tifosyl™), irinotecan (Camptosar™), bleomycin (Blenoxane™), doxorubicin (Adriamycin™; Caelyx™), mitomycin (Mitocin-C™), fludarabine (2-fluorovidarabine; 2-F-araA, Fludara™), fluorouracil (5-FU, Effluderm™), gemcitabine (Gemzar™), aminoglutethimide (Cytadren™), bicalutamide (Casodex™), celecoxib (Celebrex™), L-744832, SAHA (suberoylanilide hydroxamic acid), docetaxel (Taxotere™), epothilone D, vinblastine (Velban™, Velbe™), gefitinib (Iressa™), trastuzumab (Herceptin™), 17-AAG (17-allylamino-17-demethoxygeldanamycin), paclitaxel (Taxol™), imatinib (Gleevec™), methotrexate (methylaminopterin, amethopterin, MTX, Maxtrex™, Rheumatrex™), capecitabine (Xeloda™), vincristine (leurocristine, Oncovin™, Vincrex™), hydroxyurea (hydroxycarbamide, Droxia™, Hydrea™, Litalir™), vindesine (desacetylvinblastine amide, Eldisine™), FK-506, rapamycin, trichostatin A (TSA), callystatin A, cisplatin (cis-diamminedichloroplatinum), and discodermolide.

The efficacy of a combination of leptomycin B and specific chemotherapeutic co-agents was determined. The additive, synergistic, or antagonistic effect of the combination of leptomycin B and a co-agent was calculated for each administration regimen using Calcusyn software (Biosoft, Cambridge, United Kingdom). This software calculates a combination index using the following algorithm:
CI=[D]₁/[Dₓ]₁+[D]₂/[Dₓ]₂
where
CI is the combination index;
[D]₁ and [D]₂ are the concentrations of the two agents being tested (i.e., LMB and the co-agent) that, in combination provide a response of x % in the assay; and
[Dₓ]₁ and [Dₓ]₂ are the concentrations of the two agents that, when used alone, produce a response of x % in the assay.

A CI value of one means the combined effect of the two agents is additive. A CI value of less than one means that the two agents act synergistically. A CI value of greater than one means that the two agents act antagonistically.

The area under the curve where the fraction affected of cells (FA) ranged from 0.2 to 0.9 was calculated, to generate average CI values as shown in Table 1 for colon cancer cell line DLD-1.

TABLE 1
Combination Therapy Using Leptomycin B and a Co-Agent

| | Combination Index and Order of Administration | | |
|---|---|---|---|
| Co-Agent | Simultaneous | Co-Agent First | LMB First |
| Altretamine | 0.92 ± 0.01 | 1.11 ± 0.14 | 1.03 ± 0.004 |
| Busulfan | 1.07 ± 0.01 | 1.07 ± 0.04 | 1.14 ± 0.1 |
| Oxaliplatin | 0.59 ± 0.07 | 0.43 ± 0.11 | 0.58 ± 0.12 |
| Thiotepa | 1.12 ± 0.005 | 1.03 ± 0.14 | 1.01 ± 0.03 |
| Irinotecan | 0.92 ± 0.08 | 0.50 ± 0.001 | 0.77 ± 0.05 |
| Bleomycin | 1.41 ± 0.39 | 0.71 ± 0.04 | 0.78 ± 0.16 |
| Doxorubicin | 0.84 ± 0.12 | 0.89 ± 0.01 | 0.96 ± 0.05 |
| Mitomycin C | 1.04 ± 0.05 | 0.85 ± 0.02 | 1.04 ± 0.01 |
| Fludarabine | 1.10 ± 0.05 | 0.94 ± 0.003 | 0.70 ± 0.13 |
| Fluorouracil | 1.04 ± 0.01 | 1.12 ± 0.10 | 0.77 ± 0.06 |
| Gemcitabine | 0.97 ± 0.25 | 0.71 ± 0.31 | 1.18 ± 0.05 |
| Aminoglutethimide | 0.89 ± 0.07 | 1.02 ± 0.18 | 0.82 ± 0.05 |
| Bicalutamide | 1.02 ± 0.01 | 1.05 ± 0.02 | 1.48 ± 0.32 |
| Celecoxib | 1.02 ± 0.02 | 1.18 ± 0.23 | 1.05 ± 0.06 |
| L-744832 | 0.84 ± 0.09 | 0.86 ± 0.11 | 0.82 ± 0.05 |
| SAHA | 0.88 ± 0.04 | 1.03 ± 0.11 | 0.91 ± 0.04 |
| Docetaxel | 1.19 ± 0.14 | 0.95 ± 0.07 | 1.06 ± 0.04 |
| Epo D | 1.06 ± 0.10 | 0.92 ± 0.05 | 1.05 ± 0.03 |
| Vinblastine | 1.35 ± 0.04 | 1.10 ± 0.06 | 1.26 ± 0.04 |
| Iressa | 1.13 ± 0.02 | 1.11 ± 0.01 | 1.01 ± 0.06 |
| Trastuzumab | 0.87 ± 0.07 | 0.99 ± 0.07 | 0.84 ± 0.09 |
| 17-AAG | 1.15 ± 0.06 | 0.91 ± 0.09 | 1.13 ± 0.05 |

It can be seen from the foregoing table that in numerous instances the combinations are synergistic, although in some instances the combinations are only additive or even antagonistic.

For the administration regimen in which leptomycin B and the co-agent are administered simultaneously, preferred co-agents include altretamine, oxaliplatin, doxorubicin, aminoglutethimide, L-744832, SAHA, and trastuzumab.

For the administration regimen in which the co-agent is administered before leptomycin B, preferred chemotherapeutic co-agents include oxaliplatin, irinotecan, bleomycin, doxorubicin, mitomycin C, fludarabine, L-744832, and epothilone D.

For the administration regimen in which leptomycin B is administered before the co-agent, preferred chemotherapeutic co-agents include oxaliplatin, irinotecan, bleomycin, fludarabine, fluorouracil, aminoglutethimide, L-744832, SAHA, and trastuzumab.

The disease of cellular proliferation that can be treated according to this invention can be cancer, including, in particular embodiments, breast cancer (including metastatic breast cancer), bladder cancer, colorectal cancer (including metastatic colon cancer), non-small cell lung cancer, prostate cancer, cancers of the head and neck, cholangiocarcinoma, soft tissue sarcoma, gastric cancer, hepatocellular cancer, renal cancer, ovarian cancer, lymphoma, and brain cancer. Alternatively, the disease of cellular proliferation can be a non-cancer disease of cellular proliferation, such as psoriasis, multiple sclerosis, rheumatoid arthritis, atherosclorosis, and the like.

As used herein, “therapeutically effective amount” means that amount of active compound(s) or pharmaceutical agent(s) that elicit the biological or medicinal response in a tissue system, animal or human sought by a researcher, veterinarian, medical doctor or other clinician, which response includes alleviation of the symptoms of the disease or disorder being treated. The specific amount of active compound(s) or pharmaceutical agent(s) needed to elicit the biological or medicinal response will depend on a number of factors, including but not limited to the disease or disorder being treated, the active compound(s) or pharmaceutical agent(s) being administered, the method of administration, and the condition of the patient.

Cell Line and Reagents

Human colon adenocarcinoma cell line, DLD-1, was obtained from American Type Culture Collection (Manassas, Va.). DLD-1 cells were maintained in RPMI 1640 medium supplemented with 10% fetal bovine serum. Leptomycin B was produced by fermentation of a high producing isolate from Streptomyces sp. ATCC 39366, obtained from the American Type Culture Collection, P.O. Box 1549, Manassas, Va. 20108, USA. LMB is also available from commercial sources, such as Sigma-Aldrich. Other anti-cancer agents were either purchased commercially (e.g., from Sigma Chemical Co. (St. Louis, Mo.) or from Sequoia Research Products (Oxford, UK)).

Cell Viability Assay and Combination Effect Analysis

Cells were seeded in duplicate in 96-well microtiter plates at a density of 4,000 cells per well and allowed to attach overnight. Cells were treated with LMB and the corresponding cytotoxic drug at varying concentrations, ranging from 0.5 picomolar (“pM”) to 50 micromolar (“μM”), for 3 days. Cell viability was determined using the CellTiter-Glo luminescent cell viability assay (Promega). For the drug combination assay, cells were seeded in duplicate in 96-well plates (4,000 cells/well). After an overnight incubation, cells were treated with each drug alone and the IC₅₀ value (the concentration of drug required to inhibit cell growth by 50%) was determined. Based on the IC₅₀ values of each individual drug, combined drug treatment was designed at constant ratios of two drugs, i.e., equivalent to the ratio of their IC₅₀. Three treatment schedules were used: The first schedule used simultaneous exposure to both LMB and drug for 72 hours. In the second schedule, the cells were exposed to 24 hours of LMB. The drug was then added to the cells and incubated for 48 hours. In the third schedule, cells were exposed to the drug alone for 24 hours followed by addition of LMB for 48 hours. Cell viability was determined by the CellTiter-Glo luminescent cell viability assay. Synergism, additivity, or antagonism was determined by median effect analysis using the combination index (CI) calculated using Calcusyn (Biosoft, Cambridge, UK), as described above.

The foregoing detailed description of the invention includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various figures and descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

Further, while the present invention has been particularly described in terms of certain preferred embodiments, the invention is not limited to such preferred embodiments. Rather, the scope of the invention is defined by the appended claims.

1. A method of treating a disease of cellular proliferation, comprising administering to a subject in need of such treatment a therapeutically effective amount of a combination of leptomycin B and a chemotherapeutic co-agent.

2. A method according to claim 1, wherein the chemotherapeutic co-agent is selected from the group consisting of an anti-mitotic agent, a DNA cleaver, an alkylating agent, a DNA crosslinking agent, a DNA intercalator, an HSP90 inhibitor, a topoisomerase I inhibitor, a topoisomerase II inhibitor, an immunosuppressant, an anti-metabolite, a COX-2 inhibitor, a nucleoside (purine or pyrimidine) analog, a Ras inhibitor, a farnesyl transferase inhibitor, and a histone deacetylase inhibitor.

3. A method according to claim 1, wherein the chemotherapeutic co-agent is selected from the group consisting of altretamine, busulfan, oxaliplatin, thiotepa, irinotecan, bleomycin, doxorubicin, mitomycin, fludarabine, fluorouracil, gemcitabine, aminoglutethimide, bicalutamide, celecoxib, L-744832, SAHA, docetaxel, epothilone D, vinblastine, gefitinib, trastuzumab, 17-AAG, paclitaxel, imatinib, methotrexate, capecitabine, vincristine, hydroxyurea, vindesine, FK-506, rapamycin, trichostatin A, callystatin A, cisplatin, and discodermolide.

4. A method according to claim 1, wherein the leptomycin B and the chemotherapeutic co-agent are administered simultaneously and the chemotherapeutic agent is selected from the group consisting of altretamine, oxaliplatin, doxorubicin, aminoglutethimide, L-744832, SAHA, and trastuzumab.

5. A method according to claim 4, wherein the disease of cellular proliferation is cancer.

6. A method according to claim 5, wherein the cancer is colon cancer.

7. A method according to claim 1, wherein the leptomycin B is administered before the chemotherapeutic co-agent and the chemotherapeutic co-agent is selected from the group consisting of oxaliplatin, irinotecan, bleomycin, fludarabine, fluorouracil, aminoglutethimide, L-744832, SAHA, and trastuzumab.

8. A method according to claim 7, wherein the disease of cellular proliferation is cancer.

9. A method according to claim 8, wherein the cancer is colon cancer.

10. A method according to claim 1, wherein the chemotherapeutic co-agent is administered before the leptomycin B and the chemotherapeutic co-agent is selected from the group consisting of oxaliplatin, irinotecan, bleomycin, doxorubicin, mitomycin C, fludarabine, L-744832, and epothilone D.

11. A method according to claim 10, wherein the disease of cellular proliferation is cancer.

12. A method according to claim 11, wherein the cancer is colon cancer.
